(19) United States
(12) Patent Application Publication
(10) Pub. No.: US 2018/0192998 A1
(43) Pub. Date: Jul. 12, 2018

(54) CONTROL DEVICE OF ULTRASONIC DIAGNOSTIC APPARATUS, ULTRASONIC DIAGNOSTIC APPARATUS, CLUTTER COMPONENT REDUCING METHOD

(71) Applicant: KONICA MINOLTA, INC., Tokyo (JP)

(72) Inventors: Ikuhiro KITAGAKI, Suginami-ku Tokyo (JP); Masashi KUNITA, Midori-ku Yokohama-shi Kanagawa (JP)

(21) Appl. No.: 15/865,781

(22) Filed: Jan. 9, 2018

(30) Foreign Application Priority Data
Jan. 10, 2017 (JP) 2017-002041

(51) Int. Cl. A61B 8/00; A61B 8/08; A61B 8/06

(52) U.S. Cl. CPC A61B 8/54; A61B 8/488; A61B 8/06; A61B 8/461; A61B 8/5269

(57) ABSTRACT

A control device of an ultrasonic diagnostic apparatus includes a detector that detects a Doppler signal from a reception signal based on a reflected ultrasonic wave reflected on an internal portion of a subject, and a clutter component reducer that acquires a frequency spectrum based on the Doppler signal and reduces a clutter component from the acquired frequency spectrum based on symmetry of the clutter component which is distinguished from asymmetry of a blood flow component in the acquired frequency spectrum.

### CROSS-REFERENCE TO RELATED APPLICATION

The present application claims priority under 35 U.S.C. § 119 to Japanese Patent Application No. 2017-002041, filed on Jan. 10, 2017, the entire contents of which is incorporated herein by reference.

### BACKGROUND

### Technological Field

The present invention relates to a control device of an ultrasonic diagnostic apparatus using ultrasonic waves, an ultrasonic diagnostic apparatus, and a clutter component reducing method.

### Description of the Related Art

An ultrasonic diagnostic apparatus transmits an ultrasonic wave to a subject and analyzes information contained in a reflection echo of the ultrasonic wave to generate an internal image of the subject. Among the ultrasonic diagnostic apparatuses, the ultrasonic diagnostic apparatuses that can image a blood flow in the subject by a technique called color flow mapping (hereinafter, sometimes abbreviated to CFM) and can display a blood flow state are widely used in the whole medical fields.

The color flow mapping is a technique that uses the Doppler effect. When the blood flow is irradiated with ultrasonic waves, the Doppler effect causes the reflection echo to have a Doppler shift according to a blood flow velocity and a reflection intensity. Information on the Doppler shift is detected by quadrature detection and subjected to a high-pass filter process called a moving target indicator (MTI) filter process, an autocorrelation process, and a noise cutting process, so that information on the blood flow velocity, the power (reflection intensity), turbulence, and the like can be obtained.

In detection of the blood flow by using the color flow mapping, sometimes, clutter components (information of blood vessel walls and tissues unnecessary for blood flow detection) may appear in the blood flow at a low flow velocity. When the clutter components are mixed into a blood flow signal, it is difficult to accurately detect the blood flow. Therefore, it is desired to reduce the clutter components from the blood flow signal at a low flow velocity. As a technique for reducing clutter components from a blood flow signal, there is a technique disclosed in, for example, JP 2000-342585 A.

In detection of the blood flow by using an ultrasonic diagnostic apparatus, a high pass filter such as an MTI filter is used as one of the means for reducing the clutter. By adjusting the frequency characteristics of the filter, the clutter can be reduced, but in some cases, the performance of detection of the low-velocity blood flow is deteriorated, and thus, the blood flow cannot be detected in the region where the blood flow actually exists. On the contrary, if the frequency characteristics of the filter are adjusted so that low-velocity blood flow can be detected, the noise due to the clutter may appear largely on the image or, in the image, the blood flow may not exist in the region where the blood flow actually exists.

With the technique disclosed in JP 2000-342585 A, in order to achieve compatibility between the reduction of the clutter components and the performance of detection of the low-velocity blood flow, a process of calculating a normal distribution model and subtracting the model from the original spectrum is performed in the clutter reduction.

Since the clutter from the stationary tissue has a spectrum close to a normal distribution, the clutter can be reduced by using the technique disclosed in JP 2000-342585 A. However, for example, the clutter that is generated by movement of the tissue due to the beating of the heart in the vicinity of the heart has a spectrum that is far from the normal distribution, and thus the clutter cannot be reduced by the technique disclosed in JP 2000-342585 A and the clutter components may remain in the post-subtraction spectrum. In addition, with the technique disclosed in JP 2000-342585 A, in the case where the blood flow and the clutter power are close to each other, not only the clutter components but also the blood flow signal may be reduced. In such a case, it is difficult to accurately detect the blood flow.

### SUMMARY

An object of the present invention is to provide a control device of an ultrasonic diagnostic apparatus, an ultrasonic diagnostic apparatus, and a clutter component reducing method that can accurately detect a blood flow signal by reducing clutter components even at the time of detecting a low-velocity blood flow.

To achieve the abovementioned object, according to an aspect of the present invention, a control device of an ultrasonic diagnostic apparatus reflecting one aspect of the present invention comprises: a detector that detects a Doppler signal from a reception signal based on a reflected ultrasonic wave reflected on an internal portion of a subject; and a clutter component reducer that acquires a frequency spectrum based on the Doppler signal and reduces a clutter component from the acquired frequency spectrum based on symmetry of the clutter component which is distinguished from asymmetry of a blood flow component in the acquired frequency spectrum.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating a configuration of an ultrasonic diagnostic apparatus according to an embodiment of the present invention;

FIG. 2 is a diagram illustrating a main configuration of hardware of an ultrasonic diagnostic imaging apparatus;

FIG. 3 is a block diagram illustrating an internal configuration of a C-mode image generator;

FIG. 4A is a block diagram illustrating an internal configuration of a quadrature detection circuit;

FIG. 4B is a diagram illustrating a complex Doppler signal in a complex plane representation;

FIG. 5A is a diagram illustrating an idea of a clutter component reduction process of a clutter component reducer;

FIG. 5B is a diagram illustrating an idea of the clutter component reduction process of the clutter component reducer;

FIG. 5C is a diagram illustrating an idea of the clutter component reduction process of the clutter component reducer;

FIG. 5D is a diagram illustrating an idea of the clutter component reduction process of the clutter component reducer;

FIG. 5E is a diagram illustrating an idea of the clutter component reduction process of the clutter component reducer;

FIG. 5F is a diagram illustrating an idea of the clutter component reduction process of the clutter component reducer;

FIG. 6 is a diagram illustrating a procedure of the clutter component reduction process of the clutter component reducer;

FIG. 7 is a diagram illustrating another example of the procedure of the clutter component reduction process of the clutter component reducer;

FIG. 8 is a diagram illustrating another method of calculating a blood flow velocity V and a power P based on a frequency spectrum C(ω);

FIG. 9A is a diagram illustrating an idea of a clutter tracking process;

FIG. 9B is a diagram illustrating an idea of the clutter tracking process;

FIG. 9C is a diagram illustrating an idea of the clutter tracking process;

FIG. 9D is a diagram illustrating an idea of the clutter tracking process;

FIG. 10A is a diagram illustrating an effect of the clutter component reduction process of the ultrasonic diagnostic apparatus according to the embodiment of the present invention;

FIG. 10B is a diagram illustrating an effect of the clutter component reduction process of the ultrasonic diagnostic apparatus according to the embodiment of the present invention;

FIG. 10C is a diagram illustrating an effect of the clutter component reduction process of the ultrasonic diagnostic apparatus according to the embodiment of the present invention;

FIG. 10D is a diagram illustrating an effect of the clutter component reduction process of the ultrasonic diagnostic apparatus according to the embodiment of the present invention; and

FIG. 10E is a diagram illustrating an effect of the clutter component reduction process of the ultrasonic diagnostic apparatus according to the embodiment of the present invention.

### DETAILED DESCRIPTION OF EMBODIMENTS

Hereinafter, an ultrasonic diagnostic apparatus according to one or more embodiments of the present invention will be described with reference to the drawings. However, the scope of the invention is not limited to the disclosed embodiments. In the following description, components having the same functions and configurations are denoted by the same reference numerals, and the description thereof is omitted.

<Configuration of Ultrasonic Diagnostic Apparatus 100>

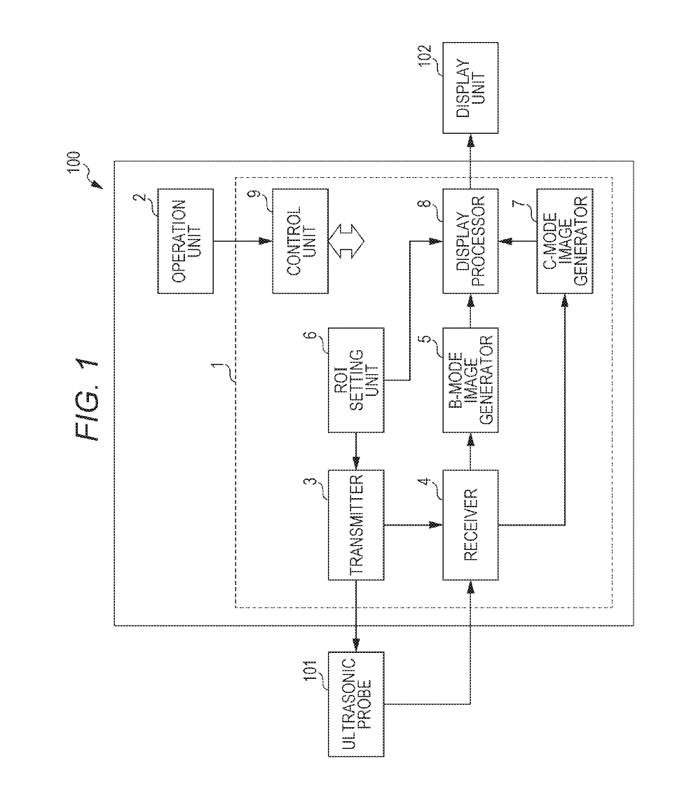

FIG. 1 is a diagram illustrating a configuration of an ultrasonic diagnostic apparatus 100 according to an embodiment of the present invention. As illustrated in FIG. 1, the ultrasonic diagnostic apparatus 100 includes a controller 1 and an operation unit 2. The controller 1 is an example of a control device according to the embodiment of the present invention. The controller 1 includes a transmitter 3, a receiver 4, a B-mode image generator 5, an ROI setting unit 6, a C-mode image generator 7, a display processor 8, and a control unit 9.

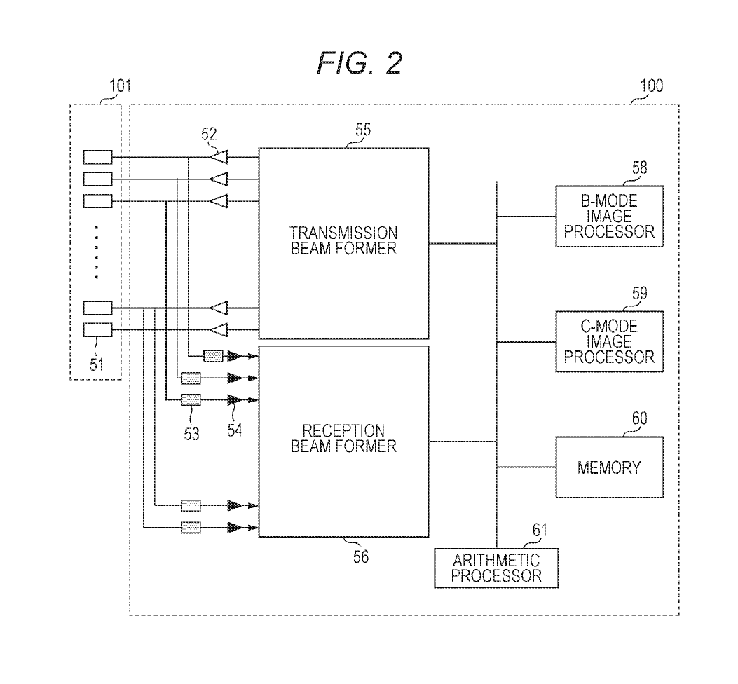

FIG. 2 is a diagram illustrating a main configuration of the hardware of the ultrasonic diagnostic apparatus 100. In terms of hardware, the ultrasonic diagnostic apparatus 100 includes, for example, a pulsar 52, an amplifier 53, an AD converter 54, a transmission beam former 55, a reception beam former 56, a B-mode image processor 58, a C-mode image processor 59, a memory 60, and an arithmetic processor 61.

An ultrasonic probe 101 includes a plurality of piezoelectric transducer elements 51 that transmit and receive ultrasonic waves and is connected to the ultrasonic diagnostic apparatus 100 through cables, connectors, wireless communication means, or the like. A plurality of the pulsars 52, the AD converters 54, and the amplifiers 53 are prepared corresponding to the number of piezoelectric transducer elements 51. The memory 60 stores a program defining procedures for realizing the functions of the components illustrated in FIG. 1 and a program defining procedures for operating each component in predetermined procedures to control the ultrasonic diagnostic apparatus 100, the ultrasonic probe 101, and the display unit 102 and to generate and display the following B-mode image and C-mode image. These programs are sequentially read from the memory 60 and executed by the arithmetic processor 61.

Each component illustrated in FIG. 1 is configured with the hardware illustrated in FIG. 2.

The transmitter 3 corresponds to the pulsar 52 and the transmission beam former 55 illustrated in FIG. 2. The receiver 4 corresponds to the amplifier 53, the AD converter 54, and the reception beam former 56 illustrated in FIG. 2. The B-mode image generator 5 corresponds to the B-mode image processor 58 illustrated in FIG. 2, the C-mode image generator 7 corresponds to the C-mode image processor 59 illustrated in FIG. 2, and the display processor 8 corresponds to the B-mode image processor 58 and the C-mode image processor 59.

On the other hand, the function of the ROI setting unit 6 is implemented by software. More specifically, the function of the ROI setting unit 6 is implemented by the arithmetic processor 61 executing a program stored in the memory 60. In other words, it can be said that the ROI setting unit 6 is configured by a program.

The above-described hardware configuration is an example, and various modifications are available. For example, the functions of the B-mode image generator 5 and the C-mode image generator 7 may be implemented by software. In addition, the functions of the transmission beam former 55 and the reception beam former 56 may be implemented by software. A personal computer including the arithmetic processor 61 and the memory 60 may be used instead of the hardware illustrated in FIG. 2.

As for the functional blocks of the controller 1, the functions of some or all of the respective functional blocks can be implemented as large scale integration (LSI) which is typically an integrated circuit. The LSIs for implementing each functional block may be individually formed into one chip, or the LSIs may be integrated into one chip so as to include some or all thereof. Although the LSI is used herein, the LSI may be called an integrated circuit (IC), a system LSI, a super LSI, or an ultra LSI depending on the degree of integration.

In addition, the method of integration is not limited to LSI, and the method of integration may be implemented by a dedicated circuit or a general-purpose processor. A field programmable gate array (FPGA) that can be programmed after LSI fabrication or a reconfigurable processor of which connection and setting of circuit cells inside the LSI can be reconfigured may be used.

Furthermore, if integrated circuit technology replacing the LSI appears due to the advancement of semiconductor technology or another derivative technology, function blocks may be integrated using the technology.

As described above, the ultrasonic probe 101 has a plurality of piezoelectric transducer elements 51 arranged in a one-dimensional direction, and each of the piezoelectric transducer elements 51 converts a transmission electric signal from the transmitter 3 described later into an ultrasonic wave to generate an ultrasonic beam. Therefore, the operator brings the ultrasonic probe 101 into contact with the surface of the subject to be measured, so that an internal portion of the subject can be irradiated with the ultrasonic beam. Then, the ultrasonic probe 101 receives reflected ultrasonic waves from the internal portion of the subject, converts the reflected ultrasonic waves into reception electric signals by the plurality of piezoelectric transducer elements 51, and supplies the reception electric signals to the receiver 4 described later.

In the embodiment of the present invention, as an example of the ultrasonic probe 101, the ultrasonic probe 101 in which a plurality of piezoelectric transducer elements 51 is arranged in a one-dimensional direction is described. However, the present invention is not limited to this example. For example, an ultrasonic probe in which a plurality of piezoelectric transducer elements is two-dimensionally arranged, an ultrasonic probe in which a plurality of piezoelectric transducer elements arranged in a one-dimensional direction swings, or the like may be used. In addition, under the control of the control unit 9, by the transmitter 3 selecting an element to be used from among the plurality of piezoelectric transducer elements 51 and changing the timing of applying a voltage to the selected element or the value of voltage for each element, it is possible to control the irradiation position and the irradiation direction of the ultrasonic beam transmitted by the ultrasonic probe 101.

In addition, the ultrasonic probe 101 may include a portion of the functions of the transmitter 3 and the receiver 4 described later. More specifically, for example, based on a control signal for generating the transmission electric signal (hereinafter, referred to as “transmission signal”) output from the transmitter 3, the ultrasonic probe 101 may generate the transmission electric signal in the ultrasonic probe 101, convert the transmission signal into an ultrasonic wave by the piezoelectric transducer element 51, convert the received reflected ultrasonic wave into a reception electric signal, and generate a reception signal described later based on the reception electric signal in the ultrasonic probe 101.

The ultrasonic probe 101 and the ultrasonic diagnostic apparatus 100 may be configured to be electrically connected to each other through a cable or the like or may be configured to transmit and receive a transmission signal and a reception signal through wireless communication. However, in the case of the configuration performing wireless communication, a communication unit needs to be included so that the ultrasonic diagnostic apparatus 100 and the ultrasonic probe 101 can communicate wirelessly.

The display unit 102 is a display device that displays an image output from the ultrasonic diagnostic apparatus 100 (a display processor 8 described later). In the embodiment, the configuration where the display unit 102 and the ultrasonic diagnostic apparatus 100 are separated from each other and the display unit 102 and the ultrasonic diagnostic apparatus 100 are connected to each other is exemplified, but the present invention is not limited thereto. More specifically, for example, the display unit 102 and the operation unit 2 described later may be integrated to constitute a touch panel.

The operation unit 2 is an operation device which receives an input from an operator and outputs a command based on the input by the operator to the ultrasonic diagnostic apparatus 100, specifically, to the control unit 9 of the controller 1. The operation unit 2 receives an operation of the operator to, for example, select a mode for displaying only the B-mode image on the display unit 102 (hereinafter, referred to as a “B mode”) or a mode for overlapping and displaying the C-mode image on the B-mode image on the display unit 102 (hereinafter, referred to as a “C mode”).

The B-mode image is an image obtained by imaging an internal tissue of the subject by displaying the amplitude intensity of the reflected ultrasonic wave with luminance. On the other hand, the C-mode image is an image in which blood flow information is displayed in color within a region of interest (ROI) specified in the B-mode image. The C-mode image is an image in which the blood flow information in the ROI is displayed, for example, with a blood flow in a direction approaching the ultrasonic probe as red components and a blood flow in a direction moving away from the ultrasonic probe as blue components. The C-mode image is overlapped and displayed on the B-mode image. In the case where the C mode is selected, the operation unit 2 receives an operation of the operator to specify the position of the ROI.

In the case where the C mode is selected, the operation unit 2 receives an operation of selecting one of a plurality of display modes such as a V mode in which the flow velocity and direction of the blood flow are displayed in color by the blood flow velocity V as the blood flow signal indicating the state of the blood flow, a P mode in which the power of the blood flow is displayed in color by the power P of the blood flow as the blood flow signal, and a dP (directional power) mode in which the power of the blood flow including the direction is displayed in color.

The transmitter 3 generates a transmission signal for transmitting the ultrasonic beam from the ultrasonic probe 101 having the piezoelectric transducer elements 51. Then, based on the generated transmission signal, the transmitter 3 supplies a high-voltage transmission electric signal from a power supply (not illustrated) to the ultrasonic probe 101 at a predetermined timing to drive the piezoelectric transducer elements 51 of the ultrasonic probe 101. As a result, the ultrasonic probe 101 converts the transmission electric signal into an ultrasonic wave and irradiates the subject with the ultrasonic beam. Hereinafter, the process of generating the transmission signal and the process of supplying the transmission electric signal to the ultrasonic probe 101, both of which are performed by the transmitter 3, are collectively referred to as a transmission process.

In the case of displaying the C-mode image, the transmitter 3 performs the transmission process for displaying the C-mode image in addition to the transmission process for displaying the B-mode image. More specifically, for example, after the electrical transmission signal for displaying the B-mode image is supplied, the electrical transmission signal for quad signal processing (QSP) for displaying the C-mode image is repeatedly supplied n (n is, for example, 6 to 12) times in the same direction (same line), with respect to all directions (all lines) of the ROI set by the ROI setting unit 6. In addition, at the time of the transmission process, the transmitter 3 designates additional information of the transmission process for the B-mode image or the transmission process for the C-mode image and supplies the additional information to the receiver 4.

The receiver 4 performs a reception process for generating a reception signal as an electrical radio frequency (RF) signal based on the reflected ultrasonic wave corresponding to the QSP. For example, the receiver 4 receives the reflected ultrasonic wave by the ultrasonic probe 101 according to the QSP, amplifies the reception electric signal converted based on the reflected ultrasonic wave, and performs A/D conversion to generate a reception signal. Next, by repeating the transmission process of the transmitter 3 and the reception process of the receiver 4 a plurality of times, the receiver 4 acquires a plurality of reception signals corresponding to a plurality of image frames.

As described above, the receiver 4 acquires additional information from the transmitter 3. If the acquired additional information is additional information for the B-mode image, the receiver 4 supplies the reception signal to the B-mode image generator 5, and if the acquired additional information is additional information for the C-mode image, the receiver 4 supplies the reception signal to the C-mode image generator 7. Hereinafter, the reception signal for generating the B-mode image is referred to as a “B-mode reception signal”, and the reception signal for generating the C-mode image is referred to as a “C-mode reception signal”.

In addition, in the embodiment of the present invention, the configuration where the receiver 4 selects whether the reception signal corresponding to the generated image frame is for the B-mode image or the C-mode image and supplies the selected signal to each block is employed, but the present invention is not limited thereto. For example, the B-mode image generator 5 and the C-mode image generator 7 may select the reception signals corresponding to the image frames generated by the receiver 4, respectively.

In addition, in the embodiment described above, the example of using the QSP in the processes of the transmitter 3 and the receiver 4 for displaying the C-mode image has been described, but the present invention is not limited thereto. For example, other transmission process and reception process corresponding to multiple parallel reception, for example, six-column parallel reception may be used.

The B-mode image generator 5 analyzes the amplitude of the B-mode reception signal mainly, and generates data (“B-mode image data”) in which the internal structure of the subject is imaged. The B-mode image data include an image signal obtained by mainly being converted into a luminance signal according to the signal intensity of the reception signal and being subjected to coordinate transformation so that the luminance signal corresponds to the orthogonal coordinate system. The B-mode image data generated by the B-mode image generator 5 is supplied to the display processor 8.

The ROI setting unit 6 sets the ROI at a desired position on the B-mode image specified by the operation of the operator on the operation unit 2. Then, the ROI setting unit 6 supplies information on the ROI set at the desired position on the B-mode image to the transmitter 3 and the display processor 8. By using the information on the ROI, the transmitter 3 performs the transmission process corresponding to the C mode on the subject within the range in which the ROI is designated.

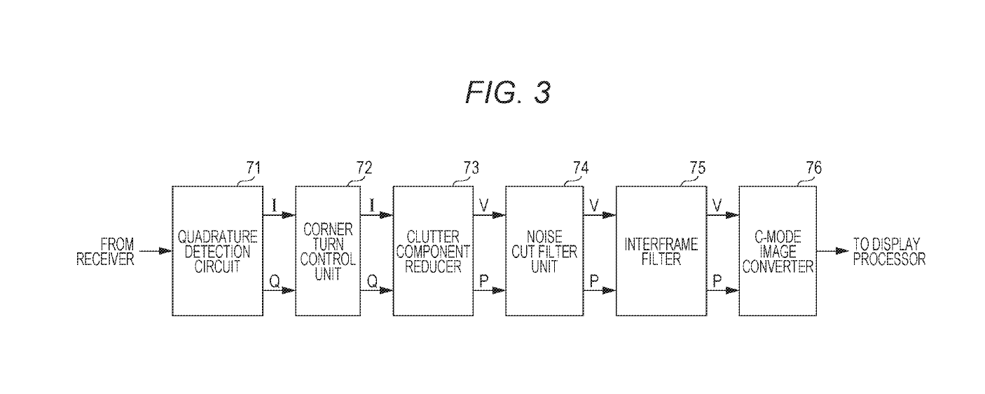

The C-mode image generator 7 generates the C-mode image based on the reception signal in the C mode acquired by the receiver 4. FIG. 3 is a block diagram illustrating the internal configuration of the C-mode image generator 7. As illustrated in FIG. 3, the C-mode image generator 7 specifically includes a quadrature detection circuit 71, a corner turn control unit 72, a clutter component reducer 73, a noise cut filter unit 74, an interframe filter 75, and a C-mode image converter 76, which respectively execute the following functions.

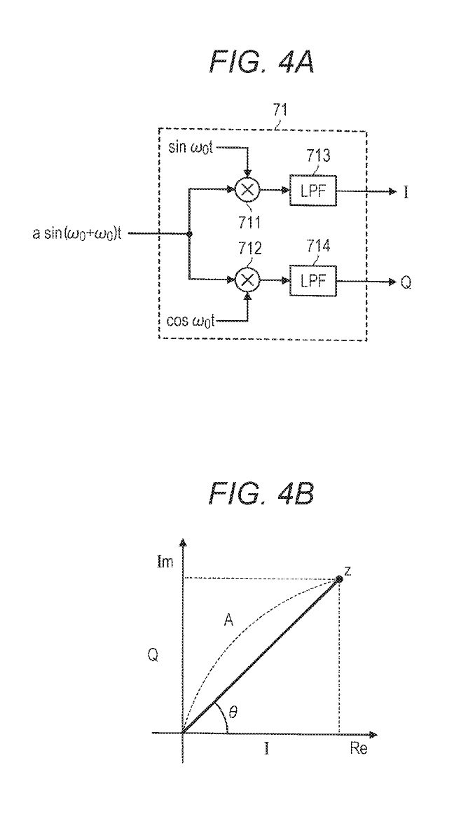

The quadrature detection circuit 71 detects the reception signal in the C mode acquired by the receiver 4 in a quadrature detection manner and calculates a phase difference between the acquired reception signal in the C mode and a reference signal to obtain a complex Doppler signal. FIG. 4A is a block diagram illustrating an internal configuration of the quadrature detection circuit 71. FIG. 4B is a diagram illustrating a complex Doppler signal in a complex plane representation.

As illustrated in FIG. 4A, the quadrature detection circuit 71 includes multipliers 711 and 712 and low pass filters (LPF) 713 and 714. Hereinafter, the reception signal (sinusoidal wave) in the C mode is represented by a·sin(ω₀+ω_{d})t. ω₀ is an angular frequency of a reference wave. The multiplier 711 multiplies the reception signal in the C mode by a reference wave (sin ω₀t). The multiplier 712 multiplies the reception signal in the C mode by a reference wave (cos ω₀t).

The LPF 713 filters a high frequency component of a multiplication result signal of the multiplier 711 and outputs the multiplication result signal as a Doppler signal I which is a real component of the complex Doppler signal. The LPF 714 filters a high frequency component of the multiplication result signal of the multiplier 712 and outputs the multiplication result signal as a Doppler signal Q which is an imaginary component of the complex Doppler signal. Then, as illustrated in FIG. 4B, the Doppler signal (I, Q) is represented as a complex Doppler signal z=I+iQ=Aeiθ (i: imaginary part) on the complex plane.

The amplitude A and the phase θ are respectively the amplitude and phase of the signal component in the band centered on the frequency f₀ corresponding to the angular frequency ω₀ of the reference wave. In this manner, the quadrature detection circuit 71 detects the amplitude A and the phase θ.

For each of the same acoustic lines, the corner turn control unit 72 arranges the Doppler signals I and Q output from the quadrature detection circuit 71 in the depth direction from the ultrasonic probe 101 to the subject and the ensemble direction of repetition number n (ensemble number) of transmission and reception of ultrasonic waves and stores the results in a memory (not illustrated). The data of the Doppler signals I and Q for each number in the ensemble direction includes a header part and a real data part, and the corner turn control unit 72 reads the real data part of the Doppler signals I and Q stored in the memory for each depth and outputs the real data part.

Herein, the Doppler signals (I, Q) contain clutter components, which are unnecessary information, in addition to the signal components of the blood flow which are necessary for generating the C-mode image. The clutter component reducer 73 performs the clutter component reduction process of extracting the clutter component from the Doppler signals (I, Q), reducing the clutter components, and outputting the blood flow velocity V and the power P with reduced clutter components. Details of the clutter component reduction process of the clutter component reducer 73 will be described later.

The noise cut filter unit 74 performs a noise cut process on the blood flow velocity V and the power P output by the clutter component reducer 73, for example, by a morphology process, or a filtering process using a keyhole filter, a spatial filter, or the like.

The interframe filter 75 smoothens the change between frames with respect to the blood flow component of each frame constituting the C-mode image corresponding to the display mode selected by the operator with the operation unit 2, among the blood flow velocities V and the powers P filtered by the noise cut filter unit 74 and leaves afterimages.

The C-mode image converter 76 converts the blood flow velocity V and the power P filtered by the interframe filter 75 into the C-mode image.

The display processor 8 performs a process of generating display image data to be displayed on the display unit 102 based on the B-mode image data output from the B-mode image generator 5, the information on the ROI output by the ROI setting unit 6, and the C-mode image data generated by the C-mode image generator 7 and displaying the display image data on the display unit 102. More specifically, in the case where the B mode is selected, the B-mode image generated by the B-mode image generator 5 is displayed on the display unit 102. In addition, in the case where the C mode is selected, the composite image data is generated by overlapping the C-mode image at the position of the ROI on the B-mode image and is displayed on the display unit 102.

<Clutter Component Reduction Process of Clutter Component Reducer 73>

Hereinafter, the clutter component reduction process of the clutter component reducer 73 will be described in detail.

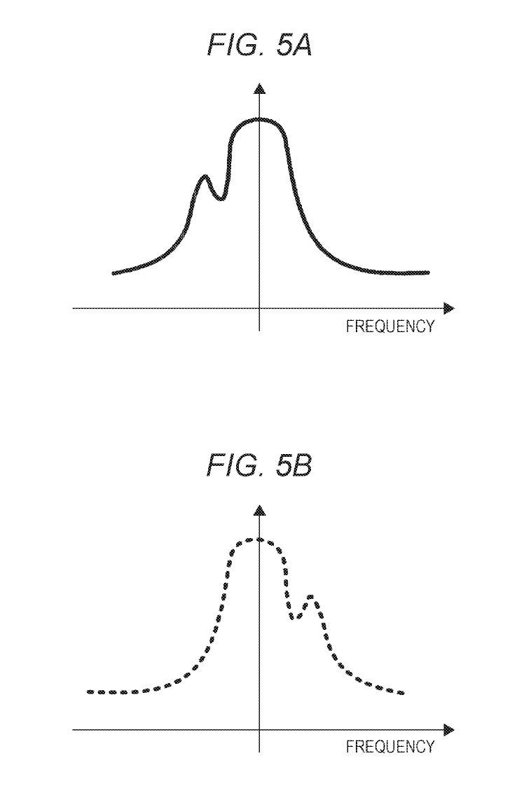

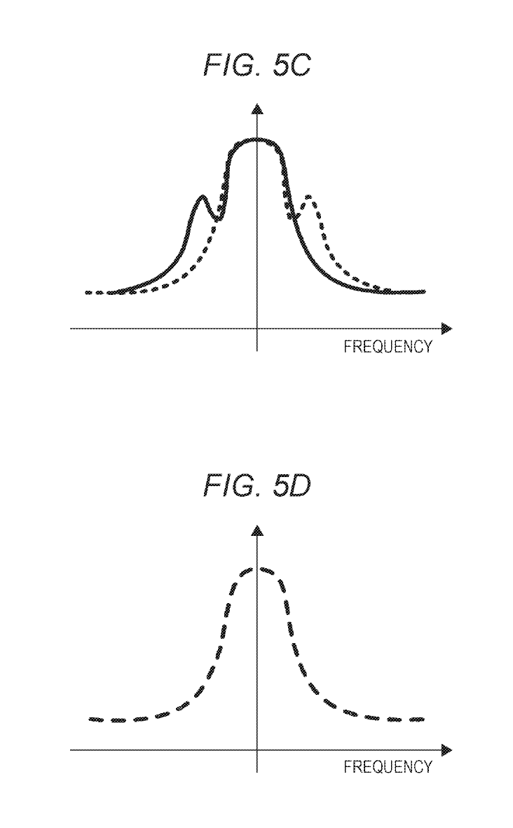

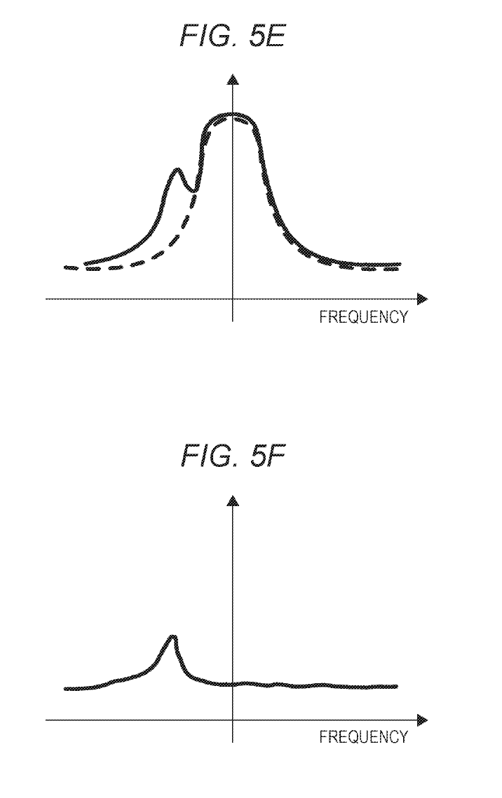

FIGS. 5A to 5F are diagrams illustrating the idea of the clutter component reduction process of the clutter component reducer 73.

First, the clutter component reducer 73 performs fast Fourier transform (FFT) and the like on the Doppler signals (I, Q) input from the corner turn control unit 72 to acquire the frequency spectrum A(ω) (FIG. 5A). The frequency spectrum A(ω) includes blood flow components and clutter components. In FIGS. 5A to 5F, the vertical axis represents the blood flow power (logarithmic expression), and the horizontal axis represents the frequency ω.

In the embodiment of the present invention, the FFT is exemplified as the conversion for the clutter component reducer 73 to acquire the frequency spectrum A(ω) from the Doppler signals (I, Q), but the present invention is not limited thereto. The clutter component reducer 73 may acquire the frequency spectrum by using another frequency conversion method.

Next, the clutter component reducer 73 reverses left and right (reverses positive and negative) of the generated frequency spectrum A(ω) (FIG. 5B). Then, the original frequency spectrum A(ω) and the left-right-reversed frequency spectrum are overlapped (FIG. 5C). In addition, in a state where the original frequency spectrum A(ω) and the reversed frequency spectrum are overlapped, one having a smaller power is extracted for each ω, and a new frequency spectrum B(ω) is generated based on the extraction result, (FIG. 5D). As illustrated in FIG. 5D, the frequency spectrum B(ω) is typically symmetrical with respect to positive and negative frequencies.

In the processes of FIGS. 5A to 5D, the clutter component reducer 73 compares A(+ω) with A(−ω) focusing on the positive/negative symmetry of the frequency spectrum A(ω) and extracts one that is smaller to generate a new frequency spectrum B(ω). This new positive/negative symmetric frequency spectrum B(ω) is considered to be a frequency spectrum corresponding to the clutter components included in the frequency spectrum A(ω) acquired based on the Doppler signals (I, Q). The reasons are as follows.

Since the clutter component has high randomness, it is considered that the average value of the frequency spectrum corresponding to the clutter components has positive/negative symmetry. Therefore, by extracting A(+ω) or A(−ω), whichever is smaller, positive and negative symmetric components of the frequency spectrum A(ω) can be extracted, and it is considered that the components correspond to the clutter.

For the reasons described above, it is possible to extract the frequency spectrum B(ω) corresponding to the clutter components in FIG. 5D. Therefore, as illustrated in FIG. 5E, the frequency spectrum B(ω) corresponding to the clutter components is subtracted from the original frequency spectrum A(ω) (more accurately, “divided”, since the vertical axis of FIGS. 5A to 5F is in a logarithmic representation), so that a frequency spectrum C(ω) corresponding to substantially only the blood flow components with the clutter components reduced can be generated (FIG. 5F).

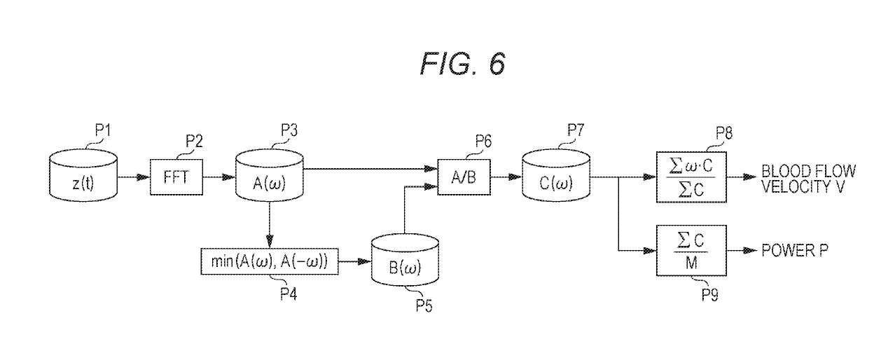

FIG. 6 is a diagram illustrating a procedure of the clutter component reduction process in the clutter component reducer 73.

When the Doppler signals (I, Q) are input from the corner turn control unit 72, the clutter component reducer 73 partitions the Doppler signals (I, Q) into data sequences having several points (process P1), and performs fast Fourier transform (FFT) based on the data sequences partitioned (process P2) to convert the Doppler signals into a frequency spectrum (process P3).

In the process P1, when the clutter component reducer 73 partitions the Doppler signals (I, Q) into data sequences, the data at several tail points of a certain data sequence and the data at several head points of the next data sequence may be overlapped. More specifically, for example, in the case where the complex Doppler signals z1, z2, z3, . . . , zM, are partitioned to the data sequences each of which has 128 points, the complex Doppler signals may be partitioned so that 120 points of each data sequence are overlapped as illustrated in the following data sequences.
(1) z1, z2, z3, . . . , z128,

(2) z9, z10, z11, . . . , z136,

(3) z17, z18, z19, . . . , z144,

(4) . . . .

By such processes, the frequency spectrum A(ω) is obtained for each data sequence (process P3).

In this manner, by overlapping the data at the several tail points of the certain data sequence and the data at the several head points of the next data sequence, it is possible to recognize the temporal change of the frequency spectrum A(ω) obtained from each data sequence in further detail. However, if the data to be overlapped is increased, it takes time to process the same amount of data. Therefore, the number of data to be overlapped should at least be set to an appropriate number by an operation of the operator, for example.

Next, for each ω, a value of A(+ω) and a value of A(−ω) are compared to extract only the one that is smaller (process P4), and the frequency spectrum B(ω) is generated based on the extracted value (process P5). The frequency spectrum B(ω) generated in the process P5 is considered to be the frequency spectrum corresponding to the clutter components as described above.

Then, the frequency spectrum B(ω) newly generated is divided by the original frequency spectrum A(ω) (process P6), and the frequency spectrum C(ω) in which the clutter components are reduced is generated (process P7). Then, the blood flow velocity V and the power P are calculated by using the frequency spectrum C(ω) in which the clutter components are reduced and only the blood flow components substantially remain (processes P8, P9).

In order to calculate the blood flow velocity V from the frequency spectrum C(ω), the following Mathematical Formula (1) is used:

$$\begin{matrix}[ Mathematical Formula 1 ] & \\ V = \frac{{\sum }_{ω = {ω}_{min}}^{{ω}_{max}} ω \cdot C ( ω )}{{\sum }_{ω = {ω}_{min}}^{{ω}_{max}} C ( ω )} & ( 1 )\end{matrix}$$

In addition, in order to calculate the power P from the frequency spectrum C(ω), the following Mathematical Formula (2) is used:

$$\begin{matrix}[ Mathematical Formula 2 ] & \\ P = \frac{{\sum }_{ω = {ω}_{min}}^{{ω}_{max}} C ( ω )}{M} & ( 2 )\end{matrix}$$

Herein, M is the number of data points of the input Doppler signal.

In this manner, the clutter component reducer 73 can calculate and output the blood flow velocity V and the power P with reduced clutter components.

<Modified Examples of Clutter Component Reduction Process>

The clutter component reduction process of the clutter component reducer 73 according to the embodiment of the present invention described above is one example, and the present invention is not limited thereto. Hereinafter, modified examples of the clutter component reduction process of the clutter component reducer 73 will be described.

### Modified Example 1

In the method described with reference to FIGS. 5A to 5F, the frequency spectrum A(ω) is reversed left and right (positive and negative), the smaller one of A(+ω) and A(−ω) is extracted, and the frequency spectrum B(ω) corresponding to clutter components is generated. In Modified Example 1, another method of generating the frequency spectrum B(ω) will be described.

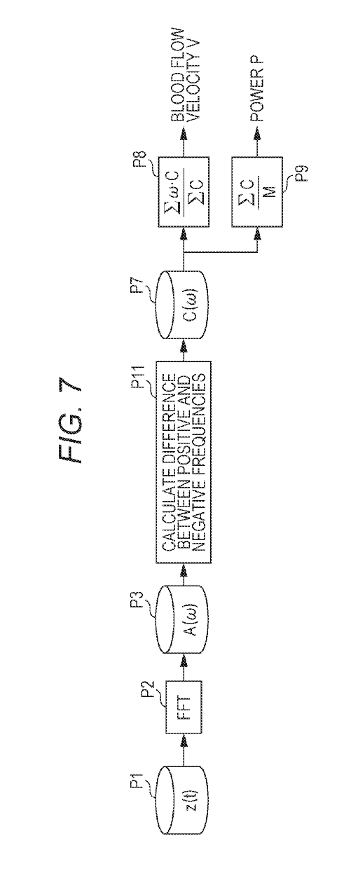

FIG. 7 is a diagram illustrating another example of the procedure of the clutter component reduction process in the clutter component reducer 73. As illustrated in FIG. 7, processes P1 to P3 and processes P7 to P9 are similar to those of the clutter component reduction process described with reference to FIG. 6, and the configuration where a process P11 is performed instead of the processes P4 to P6 illustrated in FIG. 6 is different from that of the clutter component reduction process described with reference to FIG. 6.

In the process P11, the clutter component reducer 73 calculates a comparison value d=A(ω)−A(−ω) with respect to a value of ω. Then, according to the comparison value d, a frequency spectrum C(ω) in which the clutter components are reduced as illustrated in the following Table 1 is generated.

TABLE 1

| Comparison | |
|---|---|
| value d | C (ω) |
| d ≥ 0 | d |
| d < 0 | 0 |

The frequency spectrum C(ω) thus generated is the same as the frequency spectrum C(ω) illustrated in FIG. 5F.

Therefore, according to such a method, the clutter component reducer 73 can calculate and output the blood flow velocity V and the power P with reduced clutter components. Since the clutter component reduction process described in Modified Example 1 is smaller in the number of processes than the clutter component reduction process described with reference to FIG. 6, the clutter component reduction process described in Modified Example 1 can be performed more quickly and is more preferable.

### Modified Example 2

In the clutter component reduction process described with reference to FIGS. 6 and 7, the blood flow velocity V and the power P are calculated by using Mathematical Formulas (1) and (2) based on the frequency spectrum C(ω) in which the clutter components are reduced.

In Modified Example 2, another method of calculating the blood flow velocity V and the power P based on the frequency spectrum C(ω) will be described.

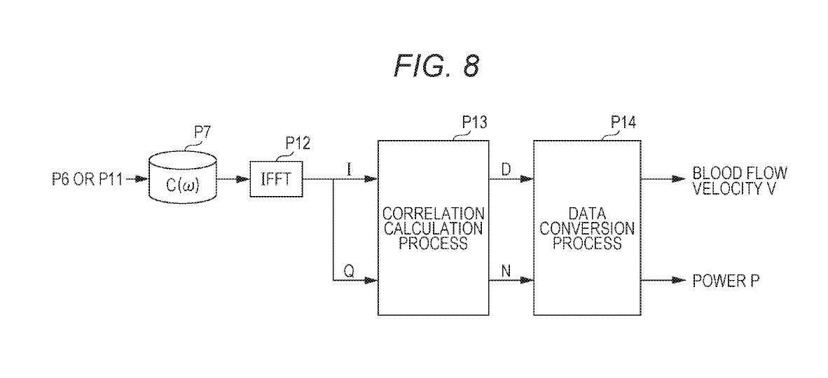

FIG. 8 is a diagram illustrating another method of calculating the blood flow velocity V and the power P based on the frequency spectrum C(ω). The processes up to the process of generating the frequency spectrum C(ω) in the process P7 are the same as those of the clutter component reduction process described with reference to FIG. 6 or FIG. 7, and thus, the description thereof will be omitted.

In the process P12, the clutter component reducer 73 performs inverse fast Fourier transform (IFFT) and the like on the generated frequency spectrum C(ω) to generate the Doppler signals (I, Q) in which the clutter components are reduced. In addition, in the case where the method other than the FFT is used as the frequency conversion, the clutter component reducer 73 should at least generate the Doppler signal by performing inverse conversion of the frequency conversion method.

In the process P13, the clutter component reducer 73 performs a correlation calculation process on the Doppler signals (I, Q) (complex Doppler signals z) in which the clutter components are reduced by using the following Mathematical Formula (3) to calculate a real part D and an imaginary part N of an average value S (an average value of a phase difference vector) of autocorrelation calculation of the Doppler signals.

$$\begin{matrix}[ Mathematical Formula 3 ] & \\ S = {\sum }_{k = 1}^{n - 1} {z}_{k}^{*} \cdot {z}_{k + 1} = D + iN & ( 3 )\end{matrix}$$

Next, in the process P14, the clutter component reducer 73 calculates the blood flow velocity V and the power P by using the following Mathematical Formulas (4) and (5) from the real part D and the imaginary part N of the average value S of the autocorrelation calculation of the Doppler signals.

$$\begin{matrix}[ Mathematical Formula 4 ] & \\ V = {tan}^{- 1} \frac{N}{D} & ( 4 ) \\ [ Mathematical Formula 5 ] & \\ P = \frac{1}{n} {\sum }_{k = 1}^{n} {\vert {z}_{k} \vert}^{2} & ( 5 )\end{matrix}$$

The blood flow velocity V and the power P thus calculated are different from the values calculated by using Mathematical Formulas (1) and (2) because the calculation method is different. However, even if the blood flow velocity V and the power P calculated by either method are used, the C-mode image converter 76 can generate the C-mode image.

### Modified Example 3

Next, in Modified Example 3 described below, an example where a clutter tracking process as described below is additionally performed in order to more accurately reduce the clutter components in the clutter component reduction process of the clutter component reducer 73 described above will be described.

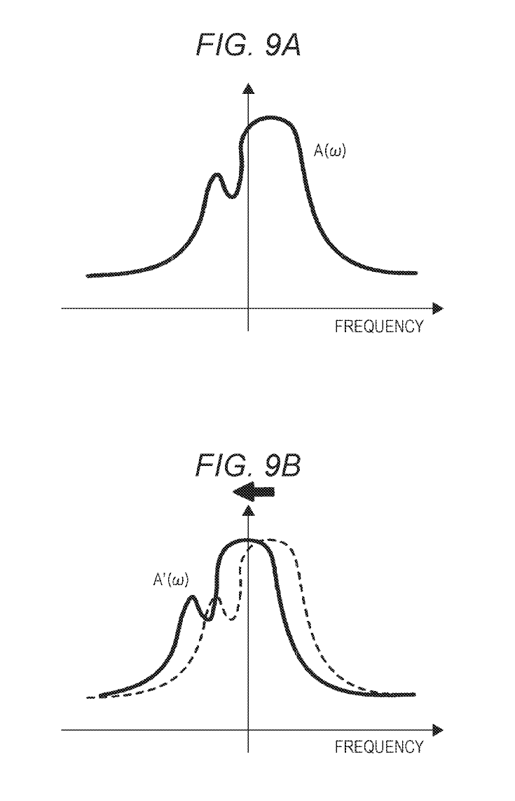

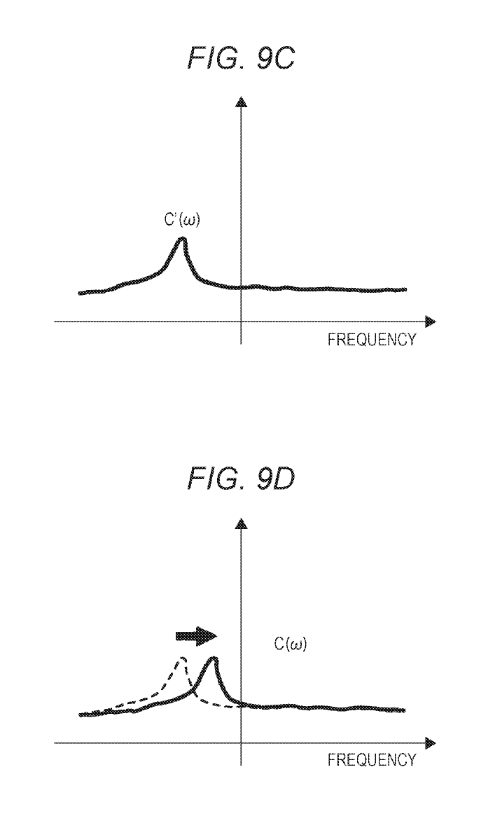

FIGS. 9A to 9D are diagrams illustrating the idea of a clutter tracking process. FIG. 9A is an example of a graph illustrating the frequency spectrum A(ω) generated in the process P3 of the clutter component reduction process described with reference to FIG. 6. In the example illustrated in FIG. 9A, the peak of the frequency spectrum A(ω) is shifted from the center of the graph.

Therefore, as illustrated in FIG. 9B, the clutter component reducer 73 takes the average of A(ω) for all frequencies ω and shifts the whole frequency spectrum so that the total values on the positive (+) side and the negative (−) side become substantially equal to each other, so that a frequency spectrum A′ (ω) is generated. In the case of FIG. 9B, the whole frequency spectrum is shifted to the left side.

Then, as illustrated in FIG. 9C, the clutter component reducer 73 generates a frequency spectrum C′(ω) in which the clutter components are reduced by the above-described method by using the frequency spectrum A′(ω).

In addition, as illustrated in FIG. 9D, the clutter component reducer 73 shifts the frequency spectrum C′(ω) by the same amount to the side opposite to FIG. 9B. In the case of FIG. 9D, the whole frequency spectrum is shifted to the right side contrary to FIG. 9B. Therefore, it is possible to generate the frequency spectrum C(ω) in which the clutter components are more appropriately reduced by using positive/negative symmetry.

### Modified Example 4

In Modified Example 4 described below, an example where a smoothing process is performed on the frequency spectrum C(ω) in which the clutter components are reduced in the clutter component reduction process of the clutter component reducer 73 described above will be described.

By performing the smoothing process on the frequency spectrum C(ω) in which the clutter components are reduced, turbulence of the calculated blood flow velocity V and power P can be reduced.

In addition, the method of the smoothing process of the clutter component reducer 73 is not limited in the present invention. For example, the clutter component reducer 73 should at least perform smoothing by, for example, taking a moving average of the frequency spectrum C(ω).

<Function and Effect>

As described above, the control device of the ultrasonic diagnostic apparatus according to the embodiment of the present invention includes: a detector that detects a Doppler signal from a reception signal based on reflected ultrasonic waves reflected on an internal portion of a subject; and a clutter component reducer that acquires a frequency spectrum based on the Doppler signal and reduces clutter components from the acquired frequency spectrum based on symmetry of the clutter components that is distinguished from asymmetry of blood flow components in the acquired frequency spectrum.

More specifically, the clutter component reducer reduces the clutter components from the frequency spectrum based on the symmetry with respect to the positive and negative frequencies of the clutter components included in the generated frequency spectrum.

More specifically, the clutter component reducer compares the intensity of the frequency spectrum A(−ω) obtained by reversing, positive and negative, the generated frequency spectrum A(ω) with the intensity of the original frequency spectrum A(ω) for each frequency ω, selects the smaller intensity to generate the frequency spectrum B(ω) of the clutter components, and subtracts the frequency spectrum B(ω) of the clutter components from the original frequency spectrum A(ω) to generate a frequency spectrum with reduced clutter components.

Alternatively, the clutter component reducer calculates, for each frequency ω of the frequency spectrum A(ω), the intensity of the frequency spectrum A(ω) with respect to the positive frequency ω and the intensity of the frequency spectrum A(−ω) with respect to the negative frequency −ω and takes the difference d=A(ω)−A(−ω). Then, the clutter component reducer generates such a new frequency spectrum C(ω) that C(ω)=d when d≥0 and C(ω)=0 when d<0 are satisfied, and sets the new frequency spectrum C(ω) as a frequency spectrum in which the clutter components are reduced from the frequency spectrum A(ω).

According to the ultrasonic diagnostic apparatus of the embodiment of the present invention having such a configuration, even in the case where clutter components are generated due to movement of the tissue such as beating of a heart, the frequency spectrum component caused by the clutter can be accurately extracted to be reduced.

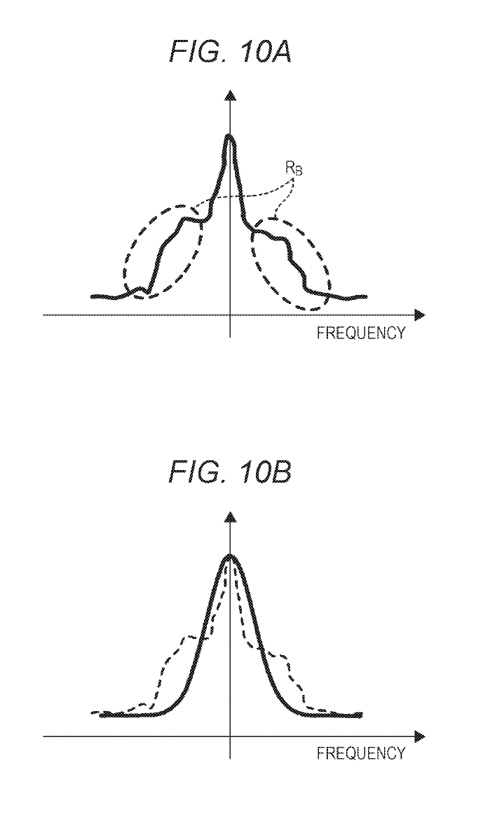

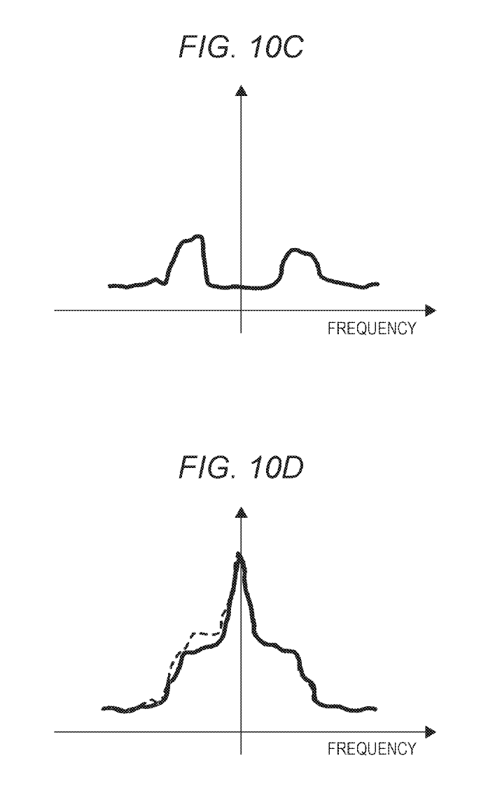

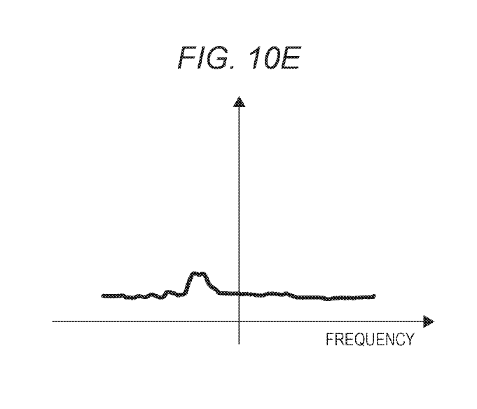

FIGS. 10A to 10E are diagrams illustrating the effects of the clutter component reduction process of the ultrasonic diagnostic apparatus according to the embodiment of the present invention. FIG. 10A is a graph illustrating a frequency spectrum including blood flow components and clutter components in the case where the clutter components are increased in a portion of frequency regions, for example, due to beating of a heart or the like. In FIG. 10A, the region R_{B} corresponds to the clutter components due to the beating of the heart.

For example, like the technique disclosed in JP 2000-342585 A, in the case where the normal distribution of the frequency spectrum is set as the clutter components, as illustrated in FIG. 10B, the normal distribution of the frequency spectrum greatly departs from the original frequency spectrum, so that the clutter components cannot be accurately extracted. For this reason, when the normal distribution of the frequency spectrum illustrated in FIG. 10B is reduced from the original frequency spectrum, as illustrated in FIG. 10C, a frequency spectrum in which the clutter components caused by beating of a heart cannot be reduced is generated.

On the other hand, in the clutter component reduction process of the ultrasonic diagnostic apparatus according to the embodiment of the present invention, since the clutter components are extracted based on the positive/negative symmetry of the clutter component as illustrated in FIG. 10D, the clutter components are accurately reduced as illustrated in FIG. 10E, so that it is possible to generate a frequency spectrum in which substantially only blood flow components are included.

Although embodiments of the present invention have been described above with reference to the drawings, the present invention is not limited to the embodiments. Various changes or modifications that can be conceived by those skilled in the art within the scope of the claims are also included in the technical scope of the present invention. In addition, the components in the above embodiments may be arbitrarily combined within the scope not deviating from the purpose of the disclosure.

The present invention is appropriate for an ultrasonic probe of an ultrasonic diagnostic apparatus using ultrasonic waves.

According to an embodiment of the present invention, it is possible to provide a control device of an ultrasonic diagnostic apparatus, an ultrasonic diagnostic apparatus, and a clutter component reducing method capable of accurately detecting a blood flow signal by reducing clutter components even at the time of detecting a low-velocity blood flow.

Although embodiments of the present invention have been described and illustrated in detail, the disclosed embodiments are made for purposes of illustration and example only and not limitation. The scope of the present invention should be interpreted by terms of the appended claims.

What is claimed is:

1. A control device of an ultrasonic diagnostic apparatus, comprising:
a detector that detects a Doppler signal from a reception signal based on a reflected ultrasonic wave reflected on an internal portion of a subject; and
a clutter component reducer that acquires a frequency spectrum based on the Doppler signal and reduces a clutter component from the acquired frequency spectrum based on symmetry of the clutter component which is distinguished from asymmetry of a blood flow component in the acquired frequency spectrum.

2. The control device according to claim 1, wherein the clutter component reducer reduces the clutter component from the frequency spectrum based on symmetry about positive and negative frequencies contained in the clutter component included in the generated frequency spectrum.

3. The control device according to claim 1, wherein the clutter component reducer generates a frequency spectrum B(ω) of the clutter component by comparing an intensity of a frequency spectrum A(−ω) obtained by reversing, positive and negative, the generated frequency spectrum A(ω) with an intensity of the original frequency spectrum A(ω) for each frequency ω and selecting the intensity of the frequency spectrum whichever is smaller, and generates the frequency spectrum in which the clutter component is reduced by subtracting the frequency spectrum B(ω) of the clutter component from the original frequency spectrum A(ω).

4. The control device according to claim 1, wherein the clutter component reducer calculates, for each frequency ω of the frequency spectrum A(ω), an intensity of the frequency spectrum A(ω) with respect to the frequency ω and an intensity of a frequency spectrum A(−ω) with respect to a frequency −ω opposite to the frequency ω, obtains a difference d=A(ω)−A(−ω), generates a new frequency spectrum C(ω) so that C(ω)=d when d≥0 and C(ω)=0 when d<0 are satisfied, and sets the new frequency spectrum C(ω) as the frequency spectrum obtained by reducing the clutter component from the frequency spectrum A(ω).

5. The control device according to claim 1, wherein the clutter component reducer generates a frequency spectrum A′(ω) by shifting the frequency spectrum in either positive or negative direction so that an average of intensities with respect to all the positive frequencies in the frequency spectrum A(ω) and an average of intensities with respect to all the negative frequencies are equal to each other and reduces the clutter component from the frequency spectrum A′(ω), and after that, shifts the frequency spectrum by the same amount in a direction opposite to the shift to generate the frequency spectrum in which the clutter components are reduced.

6. The control device according to claim 1, wherein the clutter component reducer performs a smoothing process on the frequency spectrum in which the clutter components are reduced.

7. An ultrasonic diagnostic apparatus comprising:
the control device according to claim 1; and
a display processor that displays an ultrasonic image on a display device based on the frequency spectrum in which the clutter components are reduced.

8. A clutter component reducing method to be performed by an ultrasonic diagnostic apparatus connectable to an ultrasonic probe that transmits an ultrasonic wave and receives a reflected ultrasonic wave obtained from reflection of the transmitted ultrasonic wave on a reflecting plane of a subject, the clutter component reducing method comprising:
generating a transmission signal for transmitting the ultrasonic wave from the ultrasonic probe;
generating a reception signal based on the reflected ultrasonic wave received by the ultrasonic probe;
generating a Doppler signal based on the reception signal and generating an ultrasonic image based on the Doppler signal; and
generating a frequency spectrum based on the Doppler signal and reducing a clutter component from the frequency spectrum based on symmetry about positive and negative frequencies in the frequency spectrum.
